(19) United States
(12) Patent Application Publication
(10) Pub. No.: US 2025/0308672 A1
(43) Pub. Date: Oct. 2, 2025

(54) HAPPINESS MODEL GENERATION APPARATUS, HAPPINESS CALCULATION APPARATUS, HAPPINESS CALCULATION SYSTEM, HAPPINESS MODEL GENERATION METHOD, HAPPINESS CALCULATION METHOD, AND COMPUTER READABLE MEDIUM

(71) Applicants: Mitsubishi Electric Corporation, Tokyo (JP); WASEDA UNIVERSITY, Tokyo (JP)

(72) Inventors: Takayoshi IIDA, Tokyo (JP); Futa WATANABE, Tokyo (JP); Noriko TAKAHASHI, Tokyo (JP); Kazuhiro MINAMI, Tokyo (JP); Shinichi TANABE, Tokyo (JP); Yuta FUKAWA, Tokyo (JP)

(73) Assignees: Mitsubishi Electric Corporation, Tokyo (JP); WASEDA UNIVERSITY, Tokyo (JP)

(21) Appl. No.: 19/240,124

(22) Filed: Jun. 17, 2025

Related U.S. Application Data
(63) Continuation of application No. PCT/JP2023/004844, filed on Feb. 13, 2023.

(51) Int. Cl. G16H 20/70

(52) U.S. Cl. CPC G16H 20/70

(57) ABSTRACT

A happiness model generation apparatus (100) includes a questionnaire content construction unit (110) and a happiness model construction unit (140). The questionnaire content construction unit (110) constructs as a questionnaire for model construction, a questionnaire for constructing a happiness model that deduces based on a result of administering a happiness survey questionnaire to a target person, happiness of the target person, and that describes the happiness based on three factors, a factor related to work of the target person, a first private life factor that is a factor related to a private life of the target person, and a second private life factor that is a factor among factors related to the private life of the target person that is not included in the first private life factor. The happiness model construction unit (140) constructs the happiness model based on, among questionnaire items of the questionnaire for model construction, a questionnaire item to understand happiness and a questionnaire item that correlates with the happiness.

### CROSS REFERENCE TO RELATED APPLICATION

This application is a Continuation of PCT International Application No. PCT/JP2023/004844, filed on Feb. 13, 2023, which is hereby expressly incorporated by reference into the present application.

### TECHNICAL FIELD

The present disclosure relates to a happiness model generation apparatus, a happiness calculation apparatus, a happiness calculation system, a happiness model generation method, a happiness calculation method, a happiness model generation program, and a happiness calculation program.

### BACKGROUND ART

A research is being conducted to make use of a result of visualizing happiness of a person to enhance performance of an office worker.

Non-Patent Literature 1 discloses technology in which a frequency distribution of duration of physical activity is analyzed based on an activity amount collected using a wearable device, and deducing happiness of a user based on a state of the frequency distribution analyzed.

### CITATION LIST

### Non-Patent Literature

Non-Patent Literature 1: “Measuring Happiness Using Wearable Technology—Technology for Boosting Productivity in Knowledge Work and Service Businesses—.” Hitachi Review, June/July 2015 combined issue, pp. 78-83, Hitachi, Ltd.

### SUMMARY OF INVENTION

### Technical Problem

According to the technology disclosed in Non-Patent Literature 1, there is an issue where happiness can be deduced based on a distribution of the activity amount, but a factor that affects the happiness cannot be identified.

The present disclosure aims to identify a factor that affects happiness in technology that deduces happiness.

### Solution to Problem

A happiness model generation apparatus according to the present disclosure includes:
a questionnaire content construction unit to construct as a questionnaire for model construction, a questionnaire for constructing a happiness model that is a model that deduces based on a result of administering a happiness survey questionnaire to a target person, happiness of the target person, and that is a model that describes the happiness based on three factors, a factor related to work of the target person, a first private life factor that is a factor related to a private life of the target person, and a second private life factor that is a factor among factors related to the private life of the target person that is not included in the first private life factor; and
a happiness model construction unit to construct the happiness model based on a questionnaire item to understand happiness extracted based on a result of administering the questionnaire for model construction among questionnaire items of the questionnaire for model construction, and based on a questionnaire item that correlates with the happiness extracted based on the result of administering the questionnaire for model construction among the questionnaire items of the questionnaire for model construction, wherein
the happiness survey questionnaire is a questionnaire for obtaining information to be inputted to the happiness model.

### Advantageous Effects of Invention

According to the present disclosure, a happiness model construction unit constructs a happiness model that describes happiness based on three factors. Here, happiness of a target person can be deduced using the happiness model constructed. Thus, according to the present disclosure, a factor that affects happiness can be identified in technology that deduces happiness.

### BRIEF DESCRIPTION OF DRAWINGS

FIG. 1 is a diagram illustrating an example of a configuration of a happiness model generation apparatus 100 according to Embodiment 1.

FIG. 2 is a diagram describing a happiness model 141.

FIG. 3 is a diagram illustrating an example of a configuration of a happiness calculation apparatus 200 according to Embodiment 1.

FIG. 4 is a diagram illustrating an example of a hardware configuration of the happiness model generation apparatus 100 according to Embodiment 1.

FIG. 5 is a flowchart illustrating operation of the happiness model generation apparatus 100 according to Embodiment 1.

FIG. 6 is a flowchart illustrating operation of the happiness calculation apparatus 200 according to Embodiment 1.

FIG. 7 is a diagram illustrating an example of a hardware configuration of a happiness model generation apparatus 100 according to a variation of Embodiment 1.

FIG. 8 is a diagram illustrating an example of a configuration of a happiness calculation apparatus 200 according to Embodiment 2.

FIG. 9 is a flowchart illustrating operation of the happiness calculation apparatus 200 according to Embodiment 2.

FIG. 10 is a diagram illustrating an example of a configuration of a happiness model generation apparatus 100 according to Embodiment 3.

FIG. 11 is a diagram illustrating an example of a configuration of a happiness calculation apparatus 200 according to Embodiment 3.

FIG. 12 is a flowchart illustrating operation of the happiness model generation apparatus 100 according to Embodiment 3.

FIG. 13 is a flowchart illustrating operation of the happiness calculation apparatus 200 according to Embodiment 3.

FIG. 14 is a diagram illustrating an example of a configuration of a happiness calculation system 90 according to Embodiment 4.

FIG. 15 is a flowchart illustrating operation of the happiness calculation system 90 according to Embodiment 4.

FIG. 16 is a diagram illustrating an example of a configuration of a happiness calculation apparatus 200 according to Embodiment 5.

FIG. 17 is a flowchart illustrating operation of the happiness calculation apparatus 200 according to Embodiment 5.

FIG. 18 is a diagram illustrating an example of a configuration of a happiness calculation apparatus 200 according to Embodiment 6.

FIG. 19 is a flowchart illustrating operation of the happiness calculation apparatus 200 according to Embodiment 6.

### DESCRIPTION OF EMBODIMENTS

In a description of the embodiments and in drawings, same reference signs are added to same elements and corresponding elements. A description of elements having the same reference signs added will be suitably omitted or simplified. Arrows in diagrams mainly indicate flows of data or flows of processes. “Unit” may be suitably replaced with “circuit”, “step”, “procedure”, “process”, or “circuitry”.

### Embodiment 1

The present embodiment will be described in detail below while referring to the drawings.

### ***Description of Configuration***

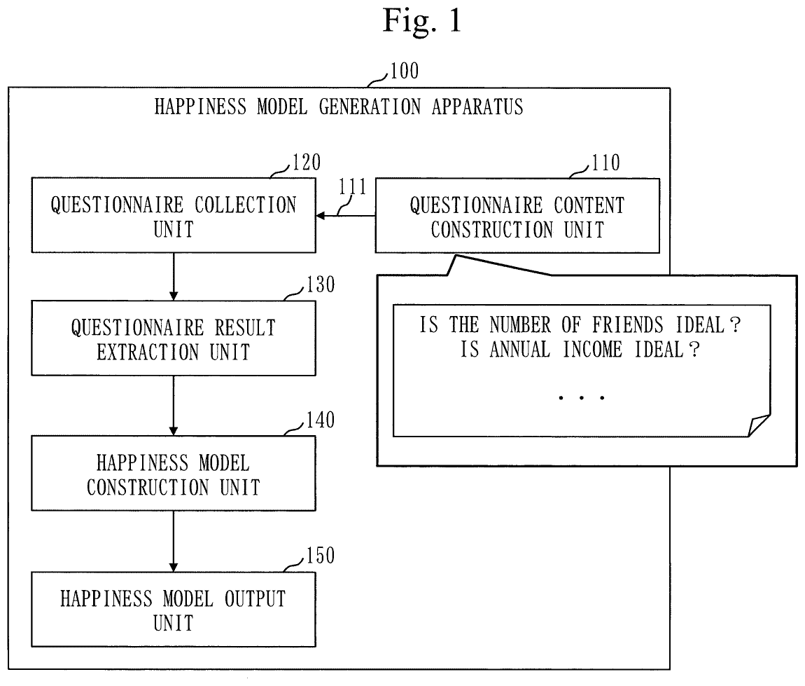

FIG. 1 illustrates an example of a configuration of a happiness model generation apparatus 100 according to the present embodiment. The happiness model generation apparatus 100, as illustrated in FIG. 1, includes a questionnaire content construction unit 110, a questionnaire collection unit 120, a questionnaire result extraction unit 130, a happiness model construction unit 140, and a happiness model output unit 150.

The questionnaire content construction unit 110 constructs a questionnaire 111.

The questionnaire content construction unit 110 may construct the questionnaire 111 using a template of the questionnaire. As a specific example, in a case where the template is to be used, the questionnaire content construction unit 110 selects question content from the template and converts a format of a question that the template indicates. Here, although a question relating to the number of friends, a question relating to annual income, or the like is a question that can also be answered quantitatively, there is an individual variation in an evaluation of happiness based on the number of friends, an evaluation of happiness based on the annual income, or the like. Therefore, by changing the question to a question with an expression, “Is xx ideal?”, a respondent is asked to answer the question imagining a deviation from a best state for the respondent. The best state may be an unrealistic ideal state or may be a realistic ideal state. Since there is a case where a question with an expression such as “Are you satisfied with xx?” makes the respondent imagine a deviation from a minimum state and is a question that makes an individual variation relating to happiness difficult to be expressed, the question is not recommended. That is, wording of a questionnaire item of a questionnaire for model construction is such wording that a respondent of the questionnaire for model construction can answer imagining a deviation from a best state for the respondent.

The questionnaire 111 is a questionnaire for constructing a happiness model 141, and is also called a questionnaire for model construction. At this time, the questionnaire content construction unit 110 selects question content and creates a question corresponding to the question content selected. The questionnaire 111 usually consists of a large number of items. The happiness model 141 is a model that deduces happiness of a target person based on a result of administering a happiness survey questionnaire to the target person, and is a model that describes the happiness based on three factors, a factor related to work of the target person, a first private life factor of the target person, and a second private life factor of the target person. The first private life factor is a factor related to a private life. The second private life factor is a factor among factors related to the private life that is not included in the first private life factor. The happiness survey questionnaire is a questionnaire for obtaining information to be inputted to the happiness model 141, and consists of a questionnaire item to understand happiness and a questionnaire item that correlates with happiness.

The questionnaire item to understand happiness and the questionnaire item that correlates with happiness are included in the questionnaire 111.

The questionnaire item to understand happiness is a questionnaire item that is administered to confirm certainty of the happiness found by the happiness model 141. The questionnaire item to understand happiness, as a specific example, consists of at least one of a questionnaire item that relates to a “life satisfaction scale” and a questionnaire item that relates to “four factors of well-being”. A specific example of the “life satisfaction scale” is stated in [Reference 1]. A specific example of the “four factors of well-being” is stated in [Reference 2].

The questionnaire item that correlates with happiness is an item that a correlation with happiness could be seen among results of administering the questionnaire for model construction.
[Reference 1] E. DIENER, et al., “The Satisfaction With Life Scale”, Journal of Personality Assessment, 1985, 49, 1, pp. 71-75
[Reference 2] Maeno, Takashi. SHIAWASE NO MEKANIZUMU JISSEN/KOUFUKUGAKU NYUUMON, Kodansha Ltd., December 2013.

The questionnaire collection unit 120 collects responses to the questionnaire 111 from a large number of people.

The questionnaire result extraction unit 130 extracts a result of the questionnaire item to understand happiness and a result of the questionnaire item that correlates with happiness from the responses to the questionnaire 111 that the questionnaire collection unit 120 collected.

The happiness model construction unit 140 uses the results that the questionnaire result extraction unit 130 extracted to construct the happiness model 141 using a method such as a covariance structure analysis and the like in a way that the results are divided into three factors, private, working, and community. Specifically, the happiness model construction unit 140 constructs the happiness model 141 based on a questionnaire item to understand happiness extracted based on a result of administering the questionnaire for model construction among questionnaire items of the questionnaire for model construction, and based on a questionnaire item that correlates with happiness extracted based on the result of administering the questionnaire for model construction among the questionnaire items of the questionnaire for model construction.

The happiness model output unit 150 outputs the happiness model 141 that the happiness model construction unit 140 constructed.

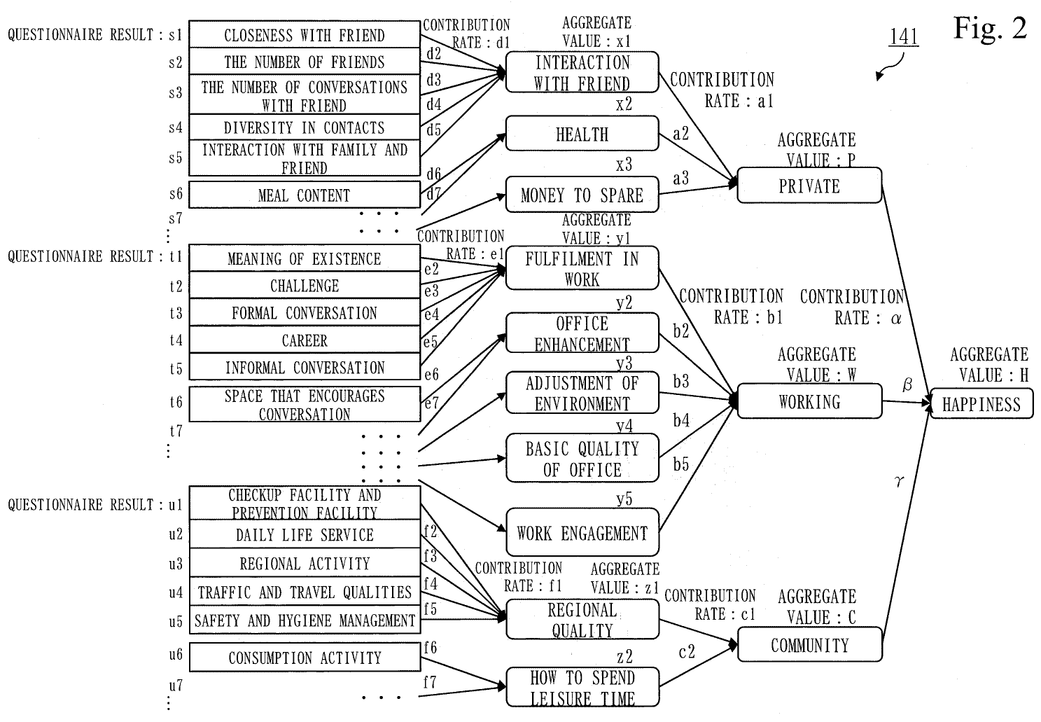

FIG. 2 illustrates a specific example of the happiness model 141. The happiness model 141 is a model that describes happiness of each individual based on the three factors, “private”, “working”, and “community”. “Working” is a factor that is directly related to work of each individual. “Private” is a factor corresponding to an event that can be changed by an effort of an individual, a daily activity of an individual or the like among factors that are not directly related to the work of each individual. “Community” is a factor that is not “private” among the factors not directly related to the work of each individual. “Community”, as a specific example, is a factor corresponding to an administrative business, a factor corresponding to a social evaluation, or a factor corresponding to content set by a different person. “Working” is the factor related to work. “Private” is the first private life factor. “Community” is the second private life factor. Questionnaire items corresponding to a leftmost column of FIG. 2 are the questionnaire items that correlate with happiness.

Each of the three factors is given a contribution rate to happiness. The happiness model 141 is generated by finding each contribution rate using the covariance structure analysis and the like based on the statistically significant number of results of the questionnaire for model construction. In the happiness model 141, each of “private”, “working”, and “community” is further decomposed into a plurality of factors, and a contribution rate is set to a relationship between each factor before decomposition and each factor after the decomposition. In the happiness model 141, a plurality of items are stratified, and a contribution rate corresponding to each item of the plurality of items is set. The number of layers of the happiness model 141 may be equal to or greater than five. When a questionnaire result is inputted to a bottommost layer of the happiness model 141, the result inputted is aggregated and happiness is finally calculated. A place of each factor may suitably be changed. As a specific example, “how to spend leisure time” may be regarded as belonging to private.

A specific example of a calculation formula of happiness is as indicated in [Mathematical Formula 1]. Here, H indicates a calculation value of happiness, P indicates a questionnaire result aggregate value in private, W indicates a questionnaire result aggregate value in “working”, C indicates a questionnaire result aggregate value in “community”, a indicates a contribution rate of the “private” factor, β indicates a contribution rate of the “working” factor, γ indicates a contribution rate of the “community” factor, x indicates a questionnaire result aggregate value of each item in “private”, y indicates a questionnaire result aggregate value of each item in “working”, z indicates a questionnaire result aggregate value of each item in “community”, a indicates a contribution rate of each item in “private”, b indicates a contribution rate of each item in “working”, c indicates a contribution rate of each item in “community”, d, e, and f indicate questionnaire contribution rates, and s, t, and u indicate questionnaire results.

$$H = α P + β W + γ C \qquad [ Mathematical Formula 1 ]$$

$$Here ,$$

$$P = a 1 x 1 + a 2 x 2 + … , W = b 1 y 1 + b 2 y 2 + … , C = c 1 z 1 + c 2 z 2 + …$$

Note that

$$x 1 = d 1 s 1 + d 2 s 2 + … , x 2 = d 6 s 6 + d 7 s7 + … , …$$

$$y 1 = e 1 t 1 + e 2 t 2 + … , y 2 = e 6 t 6 + e 7 t7 + … , …$$

$$z 1 = f 1 u 1 + f 2 u 2 + … , z 2 = f 6 u 6 + f 7 u7 + … , …$$

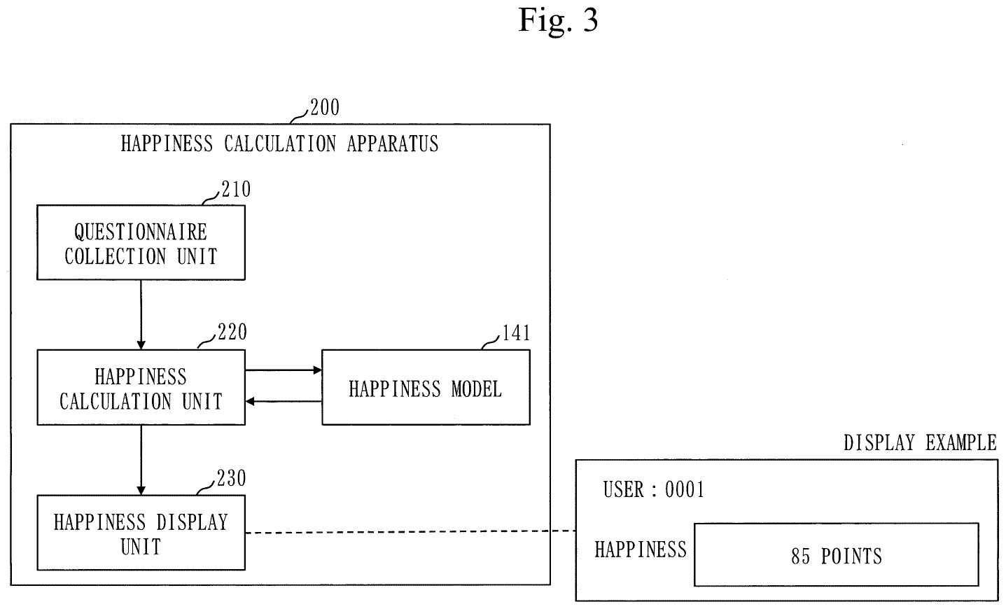

FIG. 3 illustrates an example of a configuration of a happiness calculation apparatus 200. The happiness calculation apparatus 200, as illustrated in FIG. 3, includes a questionnaire collection unit 210, a happiness calculation unit 220, and a happiness display unit 230.

The questionnaire collection unit 210 is a questionnaire for surveying happiness, and collects a questionnaire that had each individual answer. The questionnaire that the questionnaire collection unit 210 collects is also called a happiness survey questionnaire. The questionnaire collection unit 210 may collect a questionnaire result for each individual, or may collect together questionnaires that had a plurality of people answer. The happiness survey questionnaire usually differ from the questionnaire 111. The happiness survey questionnaire may consist of some questions of the questionnaire 111. Each questionnaire item of the happiness survey questionnaire, as a specific example, is an item to be answered on a 5-point scale.

The happiness calculation unit 220 calculates happiness using the questionnaire that the questionnaire collection unit 210 collected and the happiness model 141. That is, the happiness calculation unit 220 calculates the happiness of the target person using the happiness model 141 and a result of administering the happiness survey questionnaire to the target person. The happiness is a result of a comprehensive conclusion of the questionnaire result. The happiness, as a specific example, is a value that is calculated using a technique indicated in [Reference 1].

The happiness display unit 230 displays the happiness that the happiness calculation unit 220 calculated.

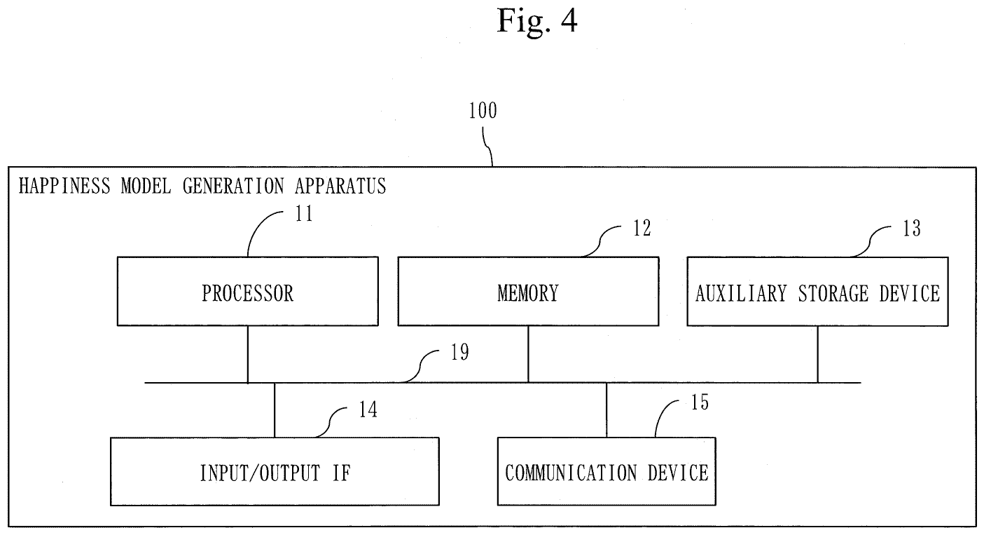

FIG. 4 illustrates an example of a hardware configuration of the happiness model generation apparatus 100 according to the present embodiment. The happiness model generation apparatus 100 consists of a computer. The happiness model generation apparatus 100 may consist of a plurality of computers.

The happiness model generation apparatus 100, as illustrated in the present diagram, is a computer that includes hardware such as a processor 11, a memory 12, an auxiliary storage device 13, an input/output IF (Interface) 14, a communication device 15, and the like. These pieces of hardware are suitably connected through a signal line 19.

The processor 11 is an IC (Integrated Circuit) that performs a calculation process, and controls hardware that the computer includes. The processor 11, as a specific example, is a CPU (Central Processing Unit), a DSP (Digital Signal Processor), or a GPU (Graphics Processing Unit).

The happiness model generation apparatus 100 may include a plurality of processors that replace the processor 11. The plurality of processors share roles of the processor 11.

The memory 12 is typically a volatile storage device, and as a specific example, is a RAM (Random Access Memory). The memory 12 is also called a main storage device or a main memory. Data stored in the memory 12 is saved in the auxiliary storage device 13 as necessary.

The auxiliary storage device 13 is typically a non-volatile storage device, and as a specific example, is a ROM (Read Only Memory), an HDD (Hard Disk Drive), or a flash memory. Data stored in the auxiliary storage device 13 is loaded into the memory 12 as necessary.

The memory 12 and the auxiliary storage device 13 may be configured integrally.

The input/output IF 14 is a port to which an input device and an output device are connected. The input/output IF 14, as a specific example, is a USB (Universal Serial Bus) terminal. Input devices, as specific examples, are a keyboard and a mouse. The output device, as a specific example, is a display.

The communication device 15 is a receiver and a transmitter. The communication device 15, as a specific example, is a communication chip or an NIC (Network Interface Card).

Each unit of the happiness model generation apparatus 100 may suitably use the input/output IF 14 and the communication device 15 when communicating with a different device and the like.

The auxiliary storage device 13 has stored a happiness model generation program. The happiness model generation program is a program that causes a computer to enable functions of each unit that the happiness model generation apparatus 100 includes. The happiness model generation program is loaded into the memory 12, and executed by the processor 11. The functions of each unit that the happiness model generation apparatus 100 includes are enabled by software.

Data used when executing the happiness model generation program, data obtained by executing the happiness model generation program and the like are suitably stored in a storage device. Each unit of the happiness model generation apparatus 100 suitably utilizes the storage device. The storage device, as a specific example, consists of at least one of the memory 12, the auxiliary storage device 13, a register in the processor 11, and a cache memory in the processor 11. There is a case where a term “data” and a term “information” have an equal meaning. The storage device may be a device that is independent of the computer.

Functions of the memory 12 and the auxiliary storage device 13 may be enabled by a different storage device.

Any of the programs according to the present description may be recorded in a computer-readable non-volatile recording medium. The non-volatile recording medium, as a specific example, is an optical disc or a flash memory. Any of the programs according to the present description may be provided as a program product.

A hardware configuration of the happiness calculation apparatus 200 is a same as the hardware configuration of the happiness model generation apparatus 100.

### ***Description of Operation***

An operation procedure of the happiness model generation apparatus 100 is equivalent to a happiness model generation method. A program that enables operation of the happiness model generation apparatus 100 is equivalent to the happiness model generation program.

An operation procedure of the happiness calculation apparatus 200 is equivalent to a happiness calculation method. A program that enables operation of the happiness calculation apparatus 200 is equivalent to a happiness calculation program.

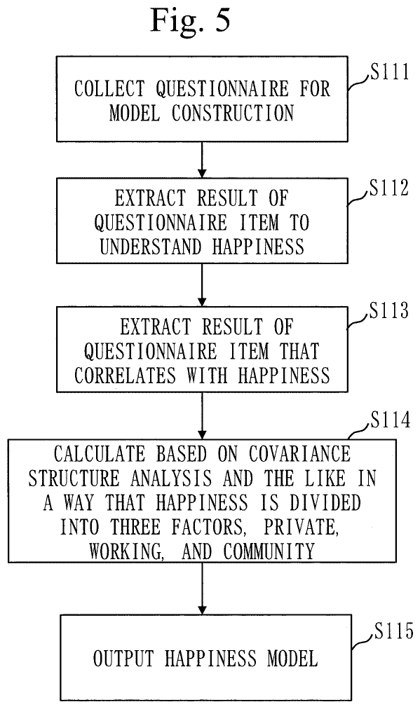

FIG. 5 is a flowchart illustrating an example of the operation of the happiness model generation apparatus 100. The operation of the happiness model generation apparatus 100 will be described using FIG. 5.

### (Step S111)

The questionnaire collection unit 120 collects the questionnaire 111.

### (Step S112)

The questionnaire result extraction unit 130 extracts a result of a questionnaire item to understand happiness from the questionnaire 111 that the questionnaire collection unit 120 collected.

### (Step S113)

The questionnaire result extraction unit 130 extracts a result of a questionnaire item that correlates with the happiness from the questionnaire 111 that the questionnaire collection unit 120 collected.

### (Step S114)

The happiness model construction unit 140 constructs the happiness model 141 by calculating based on the covariance structure analysis and the like in a way that the happiness is decomposed into three factors, private, working, and community, using the result that the questionnaire result extraction unit 130 extracted.

### (Step S115)

The happiness model output unit 150 outputs the happiness model 141 that the happiness model construction unit 140 constructed.

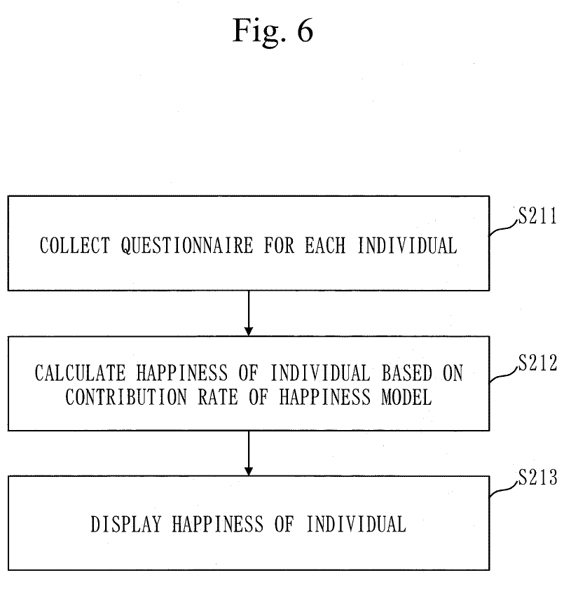

FIG. 6 is a flowchart illustrating an example of the operation of the happiness calculation apparatus 200. The operation of the happiness calculation apparatus 200 will be described using FIG. 6.

### (Step S211)

The questionnaire collection unit 210 collects a questionnaire for each individual.

### (Step S212)

The happiness calculation unit 220 calculates, using a result that the questionnaire that the questionnaire collection unit 210 collected indicates, happiness of an individual that answered the questionnaire based on a contribution rate that the happiness model 141 indicates.

### (Step S213)

The happiness display unit 230 displays the happiness of the individual that the happiness calculation unit 220 calculated.

### ***Description of Effect of Embodiment 1.***

According to the present embodiment, the happiness is deduced using the happiness model 141 that indicates the contribution rate of each factor. Consequently, according to the present embodiment, a factor that affects the happiness can be identified in the technology that deduces the happiness.

### ***Other Configurations***

### <Variation 1>

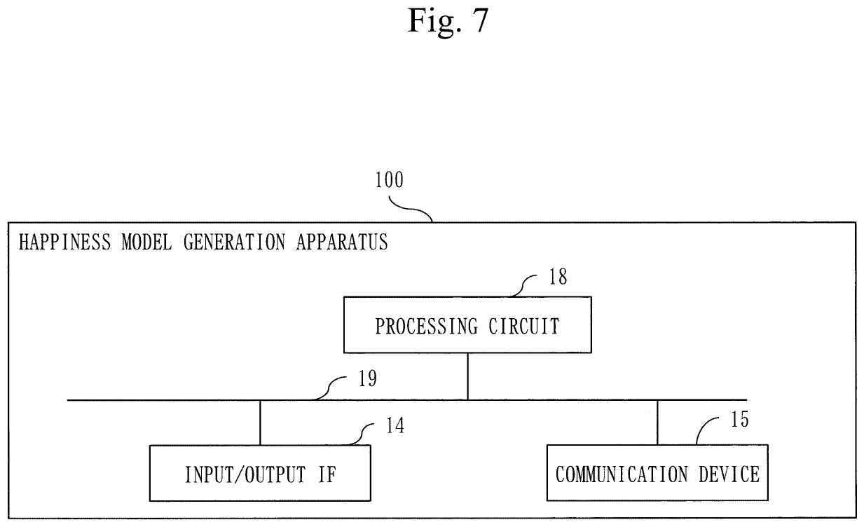

FIG. 7 illustrates an example of a hardware configuration of a happiness model generation apparatus 100 according to the present variation.

The happiness model generation apparatus 100 includes a processing circuit 18 instead of the processor 11, the processor 11 and the memory 12, the processor 11 and the auxiliary storage device 13, or the processor 11, the memory 12, and the auxiliary storage device 13.

The processing circuit 18 is hardware that enables at least a part of each unit that the happiness model generation apparatus 100 includes.

The processing circuit 18 may be dedicated hardware and may be a processor that executes a program stored in the memory 12.

In a case where the processing circuit 18 is dedicated hardware, the processing circuit 18, as a specific example, is a single circuit, a composite circuit, a programmed processor, a parallel programmed processor, an ASIC (Application Specific Integrated Circuit), an FPGA (Field Programmable Gate Array), or a combination of these.

The happiness model generation apparatus 100 may include a plurality of processing circuits that replace the processing circuit 18. The plurality of processing circuits share roles of the processing circuit 18.

In the happiness model generation apparatus 100, some of functions may be enabled by dedicated hardware and the rest of the functions may be enabled by software or firmware.

The processing circuit 18, as a specific example, is enabled by hardware, software, firmware, or a combination of these.

The processor 11, the memory 12, the auxiliary storage device 13, and the processing circuit 18 are generically called “processing circuitry”. That is, functions of each functional element of the happiness model generation apparatus 100 are enabled by the processing circuitry.

The configuration of the happiness calculation apparatus 200 may be in a same configuration as the configuration in the present variation. As for each apparatus according to other embodiments, each apparatus may be in a same configuration as the configuration in the present variation.

### Embodiment 2

Points different from the embodiment mentioned above will mainly be described below while referring to the drawings.

### ***Description of Configuration***

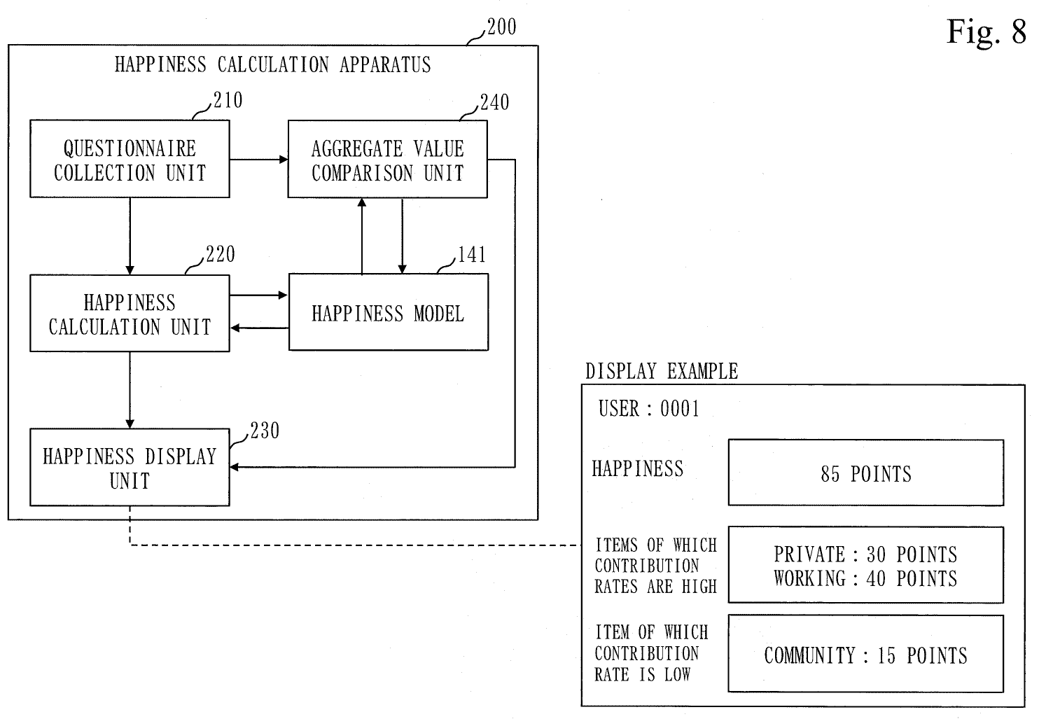

FIG. 8 illustrates an example of a configuration of a happiness calculation apparatus 200 according to the present embodiment. The happiness calculation apparatus 200 further includes an aggregate value comparison unit 240 as illustrated in FIG. 8.

The aggregate value comparison unit 240 chooses at least one of a factor with a relatively high score and a factor with a relatively low score by a threshold process and the like by comparing each aggregate value in the happiness model 141. As a specific example, the aggregate value comparison unit 240 extracts at least one of an item of which the contribution rate to the happiness that is calculated among the items that the happiness model 141 indicates is equal to or greater than a high contribution standard value, and an item of which the contribution rate to the happiness that is calculated among the items that the happiness model 141 indicates is equal to less than a low contribution standard value. Each of the high contribution standard value and the low contribution standard value may be decided on in any way.

A happiness display unit 230 according to the present embodiment displays the item chosen by the aggregate value comparison unit 240. In FIG. 8, “private” and “working” are displayed as items of which the contribution rates to the happiness are relatively high, and “community” is displayed as an item of which the contribution rate to the happiness is relatively low.

### ***Description of Operation***

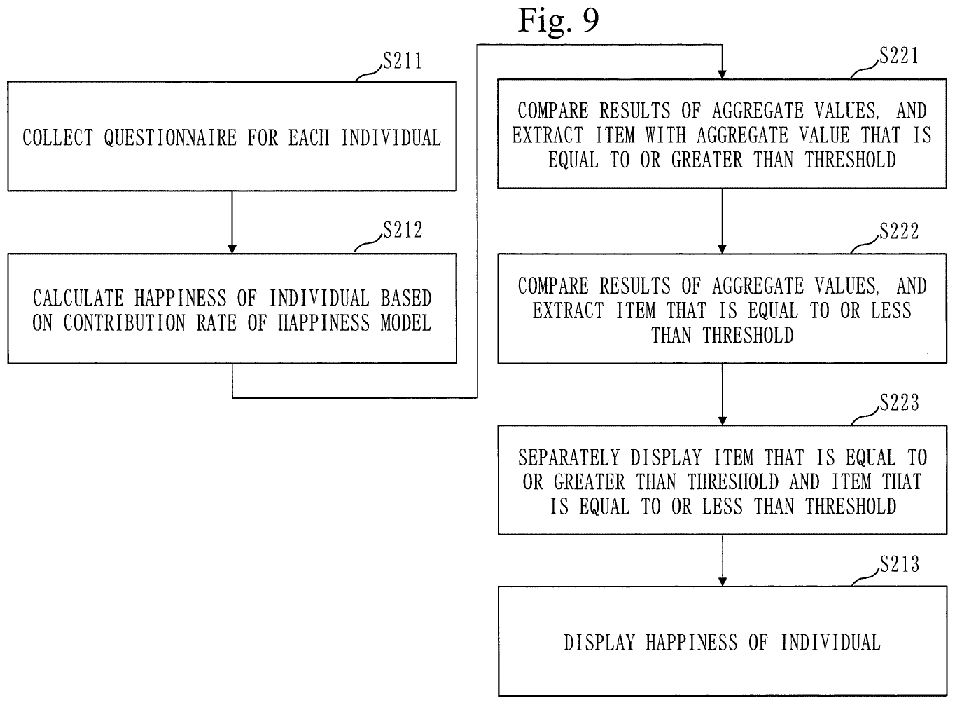

FIG. 9 is a flowchart illustrating an example of operation of the happiness calculation apparatus 200. The operation of the happiness calculation apparatus 200 will be described using FIG. 9.

### (Step S221)

The aggregate value comparison unit 240 compares results of aggregate values, and extracts an item with an aggregate value that is equal to or greater than a threshold.

### (Step S222)

The aggregate value comparison unit 240 compares the results of the aggregate values, and extracts an item with an aggregate value that is equal to or less than the threshold.

### (Step S223)

The happiness display unit 230 separately displays an item that is equal to or greater than the threshold that is extracted by the aggregate value comparison unit 240, and an item that is equal to or less than the threshold that is extracted by the aggregate value comparison unit 240.

### ***Description of Effect of Embodiment 2.***

According to the present embodiment, by comparing values calculated by utilizing a situation where the happiness is comprehensively structuralized by the happiness model 141, a factor of which a degree of contribution to the happiness is relatively high and a factor of which the degree of contribution to the happiness is relatively low can be identified.

### Embodiment 3

Points different from the embodiments mentioned above will mainly be described below while referring to the drawings.

In the present embodiment, the happiness model 141 is created for each of various individual attributes beforehand, and a most appropriate happiness model 141 is used based on the individual attribute. The individual attribute is decided on by, as a specific example, a combination of gender and age.

### ***Description of Configuration***

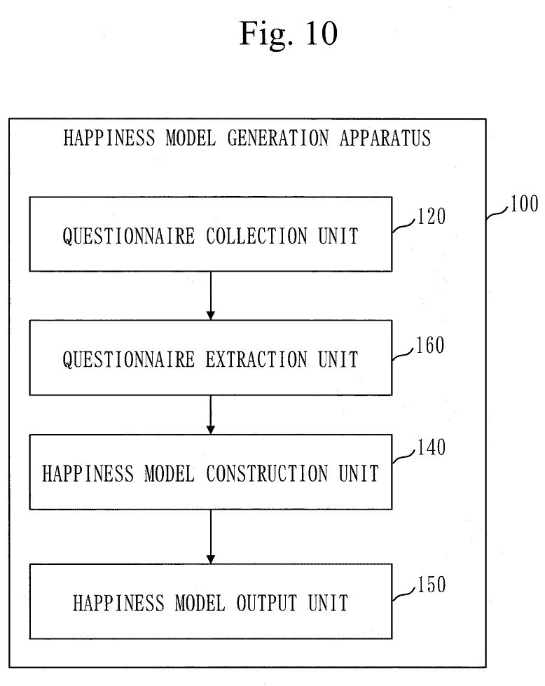

FIG. 10 illustrates an example of a configuration of a happiness model generation apparatus 100 according to the present embodiment. The happiness model generation apparatus 100, as illustrated in FIG. 10, does not include the questionnaire content construction unit 110 and the questionnaire result extraction unit 130, and further includes a questionnaire extraction unit 160.

The questionnaire extraction unit 160 extracts, for each individual attribute, a result of a questionnaire item to understand happiness and a result of a questionnaire item that correlates with the happiness.

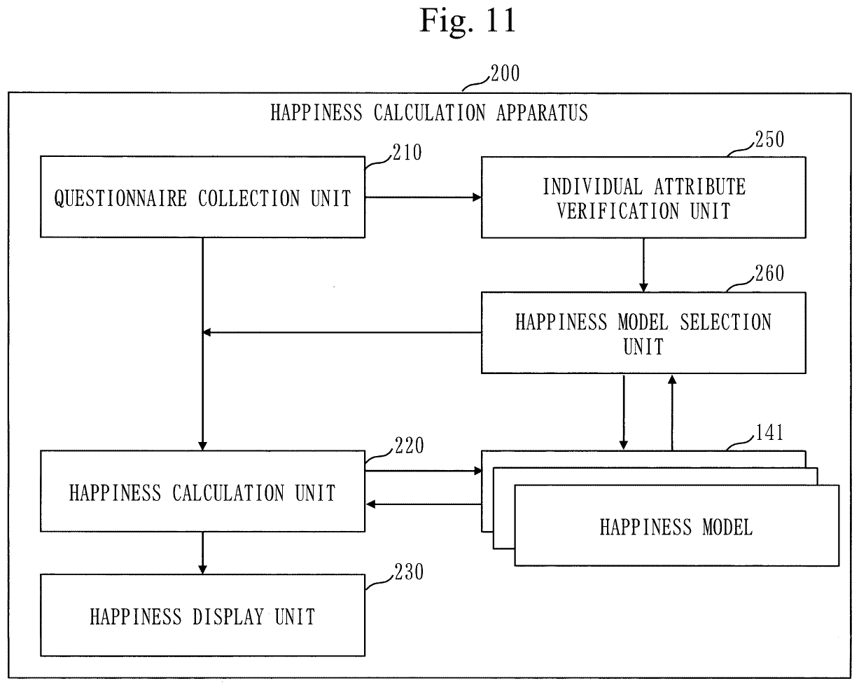

FIG. 11 illustrates an example of a configuration of a happiness calculation apparatus 200 according to the present embodiment. The happiness calculation apparatus 200 further includes an individual attribute verification unit 250 and a happiness model selection unit 260 compared with the happiness calculation apparatus 200 according to Embodiment 1. A plurality of happiness models, each of which is the happiness model 141, are prepared, and each happiness model 141 of the plurality of happiness models is a model prepared according to the individual attribute.

The individual attribute verification unit 250 reads an individual attribute stated in the questionnaire that the questionnaire collection unit 210 collected, and presents information that indicates the individual attribute read to the happiness model selection unit 260.

The happiness model selection unit 260 selects a most appropriate happiness model 141 from the plurality of happiness models based on individual attribute information presented by the individual attribute verification unit 250, and notifies the happiness calculation unit 220 of the happiness model 141 selected. That is, the happiness model selection unit 260 singles out the happiness model 141 from the plurality of happiness models according to an individual attribute of the target person

The happiness calculation unit 220 according to the present embodiment calculates happiness using the happiness model 141 notified by the happiness model selection unit 260. That is, the happiness calculation unit 220 calculates the happiness of the target person using the happiness model 141 singled out by the happiness model selection unit 260.

### ***Description of Operation***

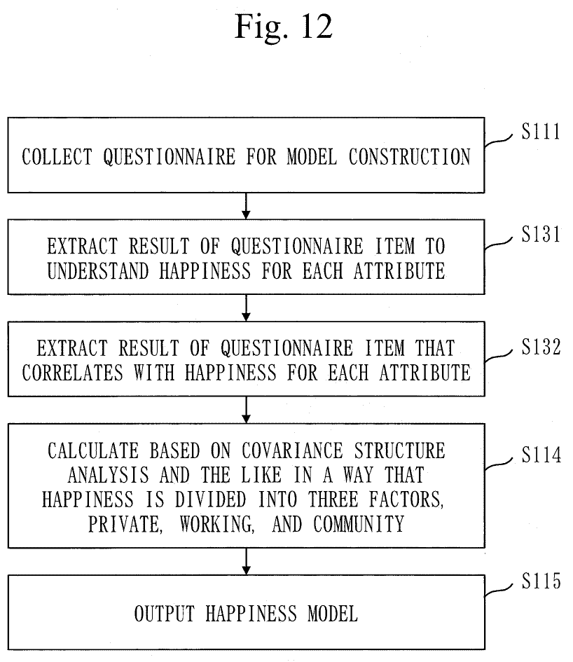

FIG. 12 is a flowchart illustrating an example of operation of the happiness model generation apparatus 100. The operation of the happiness model generation apparatus 100 will be described using FIG. 12.

### (Step S131)

The questionnaire extraction unit 160 extracts the result of the questionnaire item to understand the happiness for each attribute from the questionnaire 111 that the questionnaire collection unit 120 collected.

### (Step S132)

The questionnaire extraction unit 160 extracts the result of the questionnaire item that correlates with the happiness for each attribute from the questionnaire 111 that the questionnaire collection unit 120 collected.

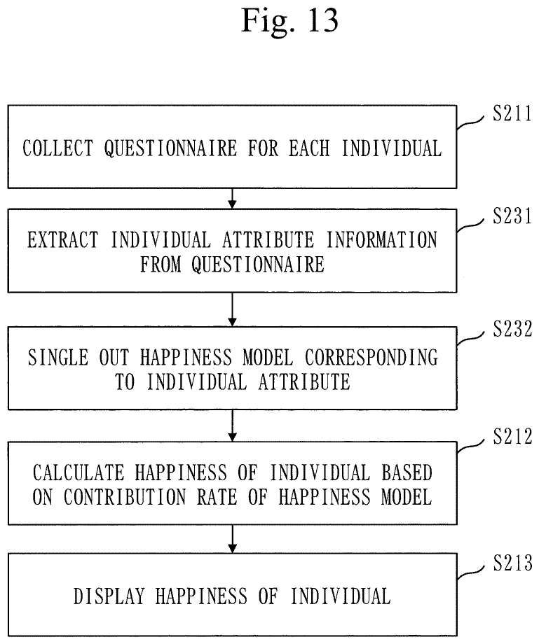

FIG. 13 is a flowchart illustrating an example of operation of the happiness calculation apparatus 200. The operation of the happiness calculation apparatus 200 will be described using FIG. 13.

### (Step S231)

The individual attribute verification unit 250 extracts the individual attribute from the questionnaire that the questionnaire collection unit 210 collected.

### (Step S232)

The happiness model selection unit 260 singles out a happiness model 141 corresponding to the individual attribute that the individual attribute verification unit 250 extracted from the plurality of happiness models.

### ***Description of Effect of Embodiment 3.***

According to the present embodiment, the happiness can be deduced more accurately since the appropriate happiness model 141 according to the individual attribute can be singled out.

### Embodiment 4

Points different from the embodiments mentioned above will mainly be described below while referring to the drawings.

In the present embodiment, a goal of an environment for a structure is set in a way that happiness is maximized, and equipment is controlled in accordance with the goal set.

### ***Description of Configuration***

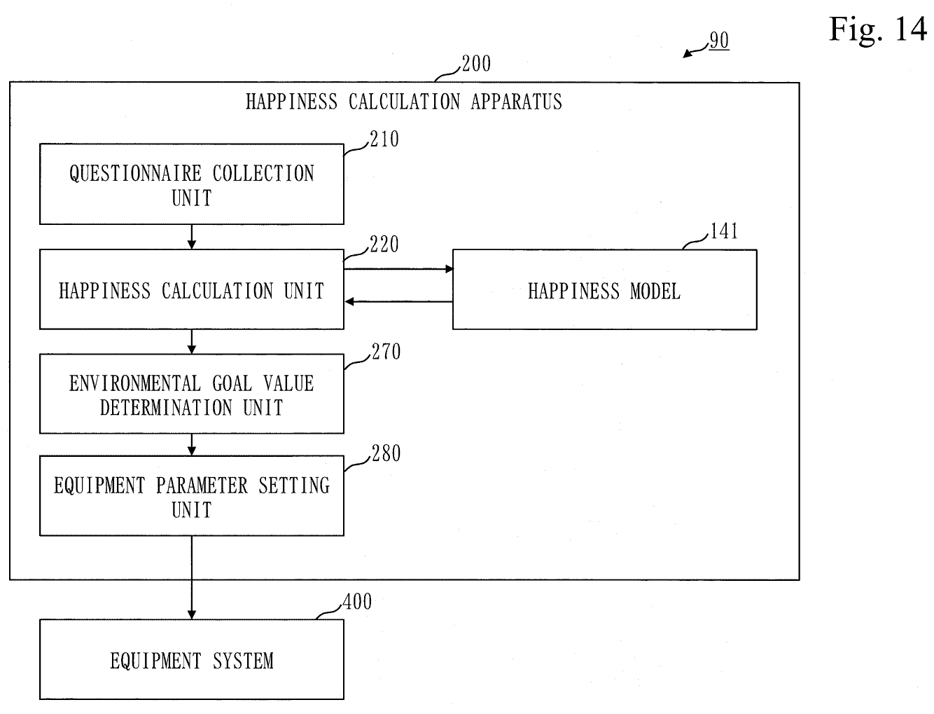

FIG. 14 illustrates an example of a configuration of a happiness calculation system 90 according to the present embodiment. The happiness calculation system 90 includes the happiness calculation apparatus 200 and an equipment system 400.

The happiness calculation apparatus 200 further includes an environmental goal value determination unit 270 and an equipment parameter setting unit 280 compared with the happiness calculation apparatus 200 according to Embodiment 1.

The equipment system 400 is a system that manages equipment that the structure and the like includes. The equipment, as a specific example, consists of a lighting device, an air conditioner, and a ventilation system. The equipment system 400 includes target equipment. The target equipment is equipment of a target structure and is equipment that is controlled according to an equipment parameter set by the equipment parameter setting unit 280. The target structure, as a specific example, is an office building.

The environmental goal value determination unit 270 determines an environmental goal value of the target structure based on the happiness calculated. As a specific example, the environmental goal value determination unit 270 confirms a questionnaire result related to a work environment from the happiness model 141, extracts a point at issue with respect to an environment from the questionnaire result confirmed, and determines an environmental goal value for improving the point at issue extracted. Specifically, in a case where a questionnaire result that relates to a degree of satisfaction with respect to a thermal environment is not good, the environmental goal value determination unit 270 determines a goal temperature of a room in a way that the thermal environment becomes appropriate. The environmental goal value determination unit 270 applies, as a specific example, a thermal model such as a PMV (Predicted Mean Vote) when calculating an appropriate goal temperature.

The equipment parameter setting unit 280 sets a parameter to be used in the equipment system 400 based on the environmental goal value determined by the environmental goal value determination unit 270. That is, the equipment parameter setting unit 280 sets the equipment parameter that controls the equipment of the target structure.

### ***Description of Operation***

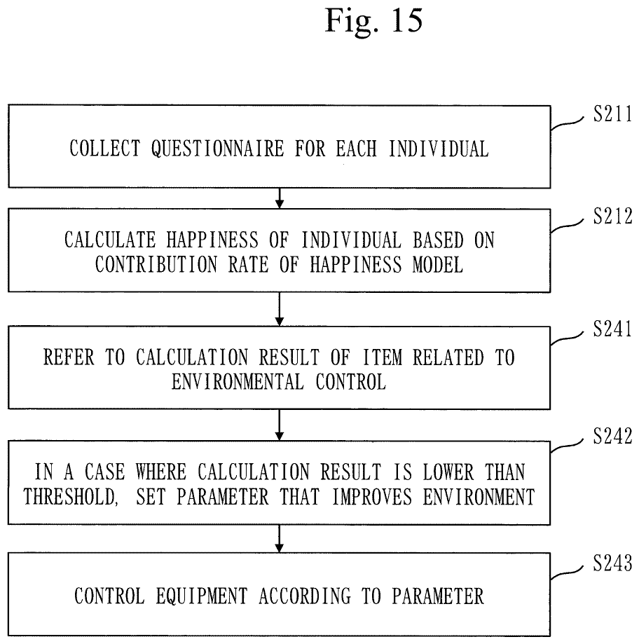

FIG. 15 is a flowchart illustrating an example of operation of the happiness calculation system 90. The operation of the happiness calculation system 90 will be described using FIG. 15.

### (Step S241)

The environmental goal value determination unit 270 determines the environmental goal value by referring to a calculation result of a factor related to environmental control in the happiness model 141.

### (Step S242)

In a case where the calculation result in the happiness model 141 is lower than the environmental goal value that the environmental goal value determination unit 270 determined, the equipment parameter setting unit 280 sets the equipment parameter in a way that the environmental goal value is attained. At this time, the equipment parameter setting unit 280 may adjust the equipment parameter in order from an equipment parameter with the contribution rate or the correlation to the happiness that is high.

### (Step S243)

The equipment system 400 controls the equipment according to the parameter set by the equipment parameter setting unit 280.

### ***Description of Effect of Embodiment 4.***

As described above, according to the present embodiment, the equipment can be controlled in a way that the happiness is equal to or greater than a certain value. Consequently, according to the present embodiment, happiness of each utilizer that utilizes the equipment can be sufficiently increased.

### Embodiment 5

Points different from the embodiments mentioned above will mainly be described below while referring to the drawings.

In the present embodiment, the happiness is calculated while reducing questionnaire items by deducing a part of the questionnaire result based on objective data.

### ***Description of Configuration***

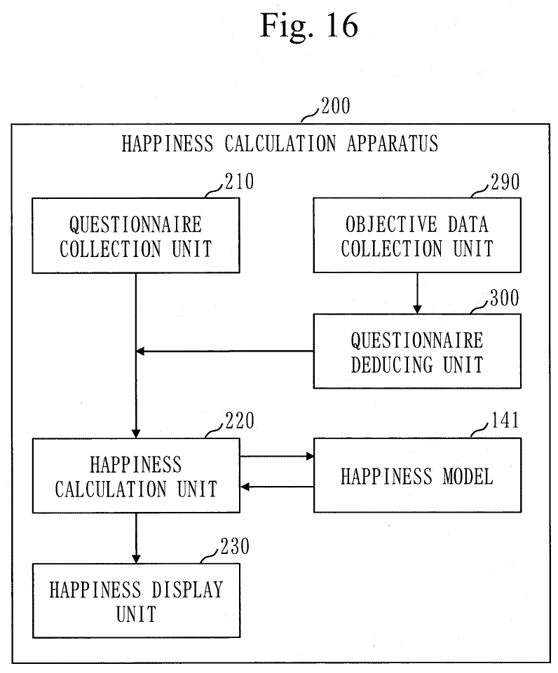

FIG. 16 illustrates an example of a configuration of a happiness calculation apparatus 200 according to the present embodiment. The happiness calculation apparatus 200, as illustrated in FIG. 16, further includes an objective data collection unit 290 and a questionnaire deducing unit 300 compared with the happiness calculation apparatus 200 according to Embodiment 1.

The objective data collection unit 290 collects the objective data. The objective data is data that has objectivity, and as a specific example, consists of body information that a wearable device obtained and temperature information that an environmental sensor obtained.

The questionnaire deducing unit 300 deduces responses to at least some of questionnaire items, the responses being responses of each individual, among questionnaire items of the happiness survey questionnaire based on the objective data that the objective data collection unit 290 collected. The questionnaire deducing unit 300, as a specific example, deduces health condition of each individual based on the objective data, deduces a degree of environmental satisfaction of each individual based on the objective data, and deduces responses to at least some of the questionnaire items based on a result deduced. At this time, the questionnaire deducing unit 300, as a specific example, applies room temperature information and a system that deduces hot and cold sensations such as the PMV.

A questionnaire that is the questionnaire item to which the questionnaire deducing unit 300 deduces the response is deleted from the happiness survey questionnaire may be used when administering the happiness survey questionnaire.

### ***Description of Operation***

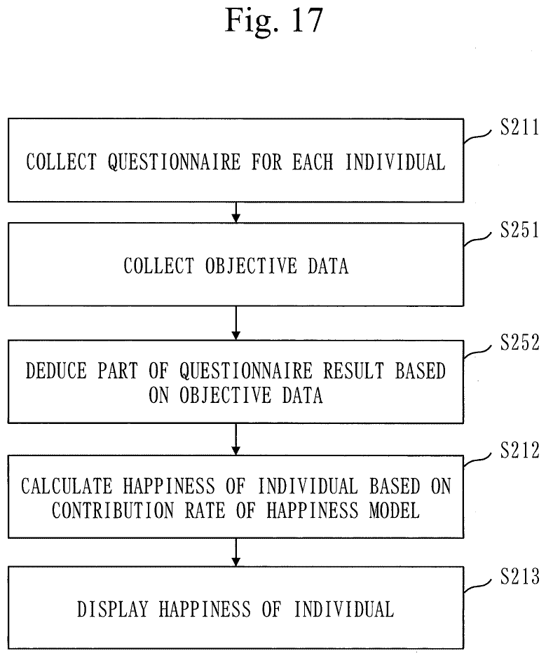

FIG. 17 is a flowchart illustrating an example of operation of the happiness calculation apparatus 200. The operation of the happiness calculation apparatus 200 will be described using FIG. 17.

### (Step S251)

The objective data collection unit 290 collects the objective data.

### (Step S252)

The questionnaire deducing unit 300 deduces responses to at least some of the questionnaire items based on the objective data that the objective data collection unit 290 collected.

### ***Description of Effect of Embodiment 5.***

According to the present embodiment, the questionnaire items can be reduced since the questionnaire result is deduced based on the objective data. Consequently, according to the present embodiment, a burden on a user of answering the questionnaire can be reduced.

### Embodiment 6

Points different from the embodiments mentioned above will mainly be described below while referring to the drawings.

### ***Description of Configuration***

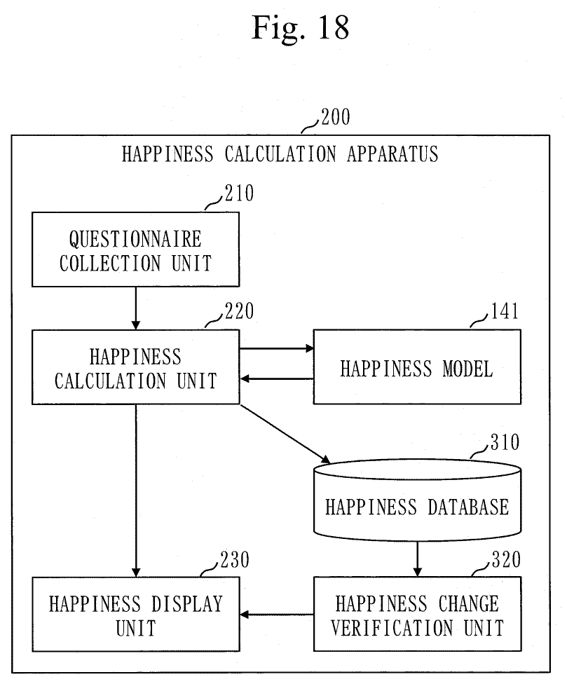

FIG. 18 illustrates an example of a configuration of a happiness calculation apparatus 200 according to the present embodiment. The happiness calculation apparatus 200, as illustrated in FIG. 18, further includes a happiness database 310 and a happiness change verification unit 320 compared with the happiness calculation apparatus 200 according to Embodiment 1.

The happiness database 310 is a database that stores time series data consisting of data that indicates the happiness calculated by the happiness calculation unit 220, and is a database that records time series happiness data. The happiness database 310 may store time series data concerning each individual.

The happiness change verification unit 320 verifies a change in the happiness based on the time series data stored in the happiness database 310.

A happiness display unit 230 according to the present embodiment may display the change in the happiness that the happiness change verification unit 320 verified.

### ***Description of Operation***

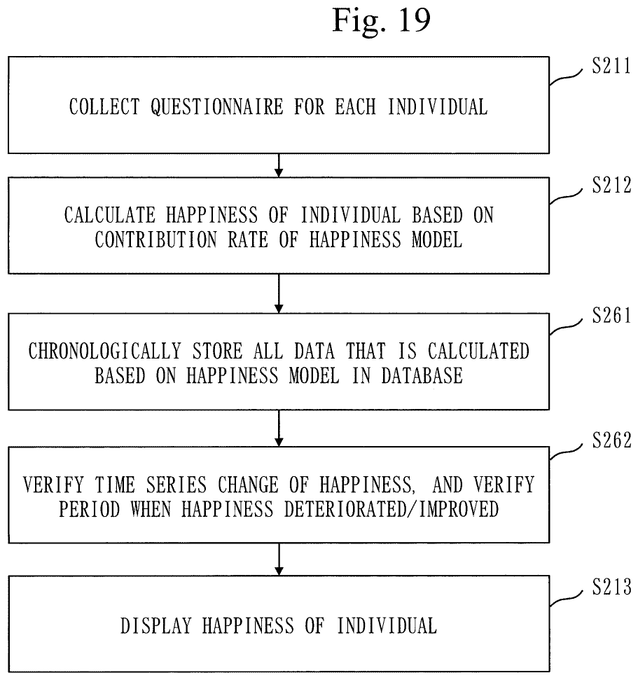

FIG. 19 is a flowchart illustrating an example of operation of the happiness calculation apparatus 200. The operation of the happiness calculation apparatus 200 will be described using FIG. 19.

### (Step S261)

The happiness calculation unit 220 stores in the happiness database 310, all data that indicates the happiness calculated as the time series data.

### (Step S262)

The happiness change verification unit 320 verifies a time series change of the happiness by referring to the data stored in the happiness database 310, and verifies at least one of a period when the happiness deteriorated and a period when the happiness improved.

### ***Description of Effect of Embodiment 6.***

As described above, according to the present embodiment, the time series change of the happiness can be confirmed.

### Other Embodiments

A free combination of each embodiment mentioned above, or a variation of any element of each embodiment, or omitting of any element in each embodiment is possible.

The embodiments are not to be limited to the embodiments indicated in Embodiments 1 to 6, and various changes are possible to be made as necessary. Procedures described using the flowcharts and the like may suitably be changed.

### REFERENCE SIGNS LIST

11: processor; 12: memory; 13: auxiliary storage device; 14: input/output IF; 15: communication device; 18: processing circuit; 19: signal line; 90: happiness calculation system; 100: happiness model generation apparatus; 110: questionnaire content construction unit; 111: questionnaire; 120: questionnaire collection unit; 130: questionnaire result extraction unit; 140: happiness model construction unit; 141: happiness model; 150: happiness model output unit; 160: questionnaire extraction unit; 200: happiness calculation apparatus; 210: questionnaire collection unit; 220: happiness calculation unit; 230: happiness display unit; 240: aggregate value comparison unit; 250: individual attribute verification unit; 260: happiness model selection unit; 270: environmental goal value determination unit; 280: equipment parameter setting unit; 290: objective data collection unit; 300: questionnaire deducing unit; 310: happiness database; 320: happiness change verification unit; 400: equipment system.

1. A happiness model generation apparatus comprising:
processing circuitry to:
construct as a questionnaire for model construction, a questionnaire for constructing a happiness model that is a model that deduces based on a result of administering a happiness survey questionnaire to a target person, happiness of the target person, and that is a model that describes the happiness based on three factors, a factor related to work of the target person, a first private life factor that is a factor related to a private life of the target person, and a second private life factor that is a factor among factors related to the private life of the target person that is not included in the first private life factor; and
construct the happiness model based on a questionnaire item to understand happiness extracted based on a result of administering the questionnaire for model construction among questionnaire items of the questionnaire for model construction, and based on a questionnaire item that correlates with the happiness extracted based on the result of administering the questionnaire for model construction among the questionnaire items of the questionnaire for model construction, wherein
the happiness survey questionnaire is a questionnaire for obtaining information to be inputted to the happiness model.

2. The happiness model generation apparatus according to claim 1, wherein
wording of the questionnaire item of the questionnaire for model construction is such wording that a respondent of the questionnaire for model construction can answer imagining a deviation from a best state for the respondent.

3. The happiness model generation apparatus according to claim 1, wherein
the processing circuitry
constructs the happiness model using a covariance structure analysis.

4. A happiness calculation apparatus comprising:
processing circuitry to:
calculate, using a happiness model that is a model that deduces based on a result of administering a happiness survey questionnaire to a target person, happiness of the target person, and that is a model that describes the happiness based on three factors, a factor related to work of the target person, a first private life factor that is a factor related to a private life of the target person, and a second private life factor that is a factor among factors related to the private life of the target person that is not included in the first private life factor, and the result of administering the happiness survey questionnaire to the target person, the happiness of the target person, wherein
the happiness survey questionnaire is a questionnaire for obtaining information to be inputted to the happiness model.

5. The happiness calculation apparatus according to claim 4, wherein
in the happiness model, a plurality of items are stratified, and a contribution rate corresponding to each item of the plurality of items is set, wherein
the processing circuitry
extracts at least one of an item of which a contribution rate to happiness that is calculated among items that the happiness model indicates is equal to or greater than a high contribution standard value, and an item of which the contribution rate to the happiness that is calculated among the items that the happiness model indicates is equal to or less than a low contribution standard value.

6. The happiness calculation apparatus according to claim 4, wherein
when a plurality of happiness models, each of which is the happiness model, are prepared, and each happiness model of the plurality of happiness models is a model prepared according to an individual attribute,
the processing circuitry
singles out a happiness model from the plurality of happiness models according to an individual attribute of the target person, and
calculates happiness of the target person using the happiness model singled out.

7. The happiness calculation apparatus according to claim 4, wherein
the processing circuitry
deduces responses to at least some of questionnaire items among questionnaire items of the happiness survey questionnaire based on objective data that is data that has objectivity.

8. The happiness calculation apparatus according to claim 4, wherein
the processing circuitry
stores time series data consisting of data that indicates happiness calculated, and
verifies a change in the happiness based on time series data stored in the happiness database.

9. The happiness calculation apparatus according to claim 4, wherein
the processing circuitry
determines an environmental goal value of a target structure based on happiness calculated, and
sets an equipment parameter that controls equipment of the target structure based on the environmental goal value determined.

10. A happiness calculation system comprising:
the happiness calculation apparatus according to claim 9; and
a target equipment that is equipment of the target structure and is equipment that is controlled according to an equipment parameter set.

11. A happiness model generation method comprising:
constructing as a questionnaire for model construction, a questionnaire for constructing a happiness model that is a model that deduces based on a result of administering a happiness survey questionnaire to a target person, happiness of the target person, and that is a model that describes the happiness based on three factors, a factor related to work of the target person, a first private life factor that is a factor related to a private life of the target person, and a second private life factor that is a factor among factors related to the private life of the target person that is not included in the first private life factor; and
constructing the happiness model based on a questionnaire item to understand happiness extracted based on a result of administering the questionnaire for model construction among questionnaire items of the questionnaire for model construction, and based on a questionnaire item that correlates with the happiness extracted based on the result of administering the questionnaire for model construction among the questionnaire items of the questionnaire for model construction, wherein
the happiness survey questionnaire is a questionnaire for obtaining information to be inputted to the happiness model.

12. A happiness calculation method comprising:
calculating, using a happiness model that is a model that deduces based on a result of administering a happiness survey questionnaire to a target person, happiness of the target person, and that is a model that describes the happiness based on three factors, a factor related to work of the target person, a first private life factor that is a factor related to a private life of the target person, and a second private life factor that is a factor among factors related to the private life of the target person that is not included in the first private life factor, and the result of administering the happiness survey questionnaire to the target person, the happiness of the target person, wherein
the happiness survey questionnaire is a questionnaire for obtaining information to be inputted to the happiness model.

13. A non-transitory computer readable medium storing a happiness model generation program that causes a happiness model generation apparatus that is a computer to execute:
a questionnaire content construction process to construct as a questionnaire for model construction, a questionnaire for constructing a happiness model that is a model that deduces based on a result of administering a happiness survey questionnaire to a target person, happiness of the target person, and that is a model that describes the happiness based on three factors, a factor related to work of the target person, a first private life factor that is a factor related to a private life of the target person, and a second private life factor that is a factor among factors related to the private life of the target person that is not included in the first private life factor; and
a happiness model construction process to construct the happiness model based on a questionnaire item to understand happiness extracted based on a result of administering the questionnaire for model construction among questionnaire items of the questionnaire for model construction, and based on a questionnaire item that correlates with the happiness extracted based on the result of administering the questionnaire for model construction among the questionnaire items of the questionnaire for model construction, wherein
the happiness survey questionnaire is a questionnaire for obtaining information to be inputted to the happiness model.

14. A non-transitory computer readable medium storing a happiness calculation program that causes a happiness calculation apparatus that is a computer to execute:
a happiness calculation process to calculate, using a happiness model that is a model that deduces based on a result of administering a happiness survey questionnaire to a target person, happiness of the target person, and that is a model that describes the happiness based on three factors, a factor related to work of the target person, a first private life factor that is a factor related to a private life of the target person, and a second private life factor that is a factor among factors related to the private life of the target person that is not included in the first private life factor, and the result of administering the happiness survey questionnaire to the target person, the happiness of the target person, wherein
the happiness survey questionnaire is a questionnaire for obtaining information to be inputted to the happiness model.
